(19) United States
(12) Patent Application Publication
(10) Pub. No.: US 2009/0312756 A1
(43) Pub. Date: Dec. 17, 2009

(54) IRRIGATED ABLATION CATHETERS

(75) Inventors: Randall L. Schlesinger, San Mateo, CA (US); Eric A. Schultheis, Los Altos, CA (US)

Correspondence Address: VISTA IP LAW GROUP LLP, 12930 Saratoga Avenue, Suite D-2, Saratoga, CA, 95070, US

(73) Assignee: Hansen Medical, Inc., Mountain View, CA (US)

(21) Appl. No.: 12/486,380

(22) Filed: Jun. 17, 2009

Related U.S. Application Data
(60) Provisional application No. 61/132,362, filed on Jun. 17, 2008.
(60) Provisional application No. 61/079,774, filed on Jul. 10, 2008.

(51) Int. Cl. A61B 18/14

(52) U.S. Cl. 606/41

(57) ABSTRACT

An apparatus for ablating tissue includes an elongate flexible member having a proximal end and a distal end. The elongate flexible member includes an irrigation lumen disposed between the proximal end and the distal end of the elongate flexible member. The irrigation lumen is configured to deliver irrigation fluid from the proximal portion of the elongate flexible member to the distal portion of the elongate flexible member. An ablation member is coupled to the distal end of the elongate flexible member. The ablation member is in fluid communication with the irrigation lumen. The ablation member comprises of a shell having a side wall and a distal wall. The side wall and distal walls of the shell define a cavity or reservoir for containing the irrigation fluid. The side wall includes a plurality of ports for dispensing fluid from the reservoir. A thermocouple is disposed from the proximal end of the elongate flexible member to a distal portion of the elongate member, wherein a distal tip of the thermocouple is positioned proximal to the irrigation reservoir and the thermocouple is electrically isolated from the ablation member.

### CROSS-REFERENCE TO RELATED APPLICATIONS

The present application claims the benefit of priority under 35 U.S.C. section 119 to U.S. Provisional Application No. 61/132,362 filed on Jun. 17, 2008, and U.S. Provisional Application No. 61/079,774 filed on Jul. 10, 2008, contents of which are incorporated herein by reference as though set forth in full.

### FIELD OF INVENTION

The present invention relates generally to minimally invasive surgical instruments, such as ablation catheters, and more particularly to irrigated ablation catheter and the apparatus and methods for monitoring and/or controlling the temperature and/or cooling of the distal tip of the ablation catheter.

### BACKGROUND

In various medical applications where electrical energy, such as radio frequency (RF) electrical current, is delivered into a tissue of a patient through a small surface on an electrode, it may be desirable to monitor and control the temperature of the electrode to prevent overheating of the tissue. Many conventional ablation catheters lack effective means to monitor and control the temperature of the electrode to prevent overheating and charring of the tissue, especially when a large amount of current is delivered through the electrode to the tissue. Therefore, it would be desirable to provide the apparatuses and methods to cool the electrode at the distal end of an ablation catheter to prevent overheating and charring of the tissue as it is being ablated.

### SUMMARY

Embodiments of the present invention include various apparatuses having an elongate body configured with an ablation member for delivering electrical energy into tissue structures of a patient. The apparatuses also include a fluid lumen configured to deliver cooling fluid for cooling the ablation member to prevent overheating of tissue structures. Embodiments of the present invention also include various configurations for directing fluid (e.g., saline, etc.) out of the ablation member or distal portion of the elongate body to prevent overheating of tissue structures.

An apparatus for ablating tissue in accordance with one embodiment of the present invention includes an elongate flexible member having a proximal end and a distal end. The elongate flexible member includes an irrigation lumen disposed between the proximal end and the distal end of the elongate flexible member. The irrigation lumen may be configured to deliver irrigation fluid from the proximal portion of the elongate flexible member to the distal portion of the elongate flexible member. An ablation member may be coupled to the distal end of the elongate flexible member. The ablation member may be in fluid communication with the irrigation lumen. The ablation member may be comprised of a shell having a side wall and a distal wall. The side wall and distal walls of the shell may define a cavity or reservoir for containing the irrigation fluid. The side wall may include a plurality of ports for dispensing fluid from the reservoir. A thermocouple may be disposed along elongate flexible member from the proximal end of the elongate flexible member to a distal portion of the elongate member. A distal tip of the thermocouple may be positioned proximal to the irrigation reservoir and the thermocouple may be electrically isolated from the ablation member. The thermocouple may be configured to monitor the temperature of the ablation member. Irrigation fluid may be used to control or regulate the temperature of the ablation member.

In another embodiment of the present invention, a medical instrument includes a steerable irrigated ablation catheter configured for ablating tissue structures inside a patient. The steerable irrigated ablation catheter includes a proximal end and a distal ablation tip, wherein a fluid reservoir may be located in distal portion of the steerable irrigated catheter. A thermocouple may be positioned within the steerable irrigated ablation catheter to monitor the temperature of the distal portion of the catheter. The thermocouple may be disposed along the body of the irrigated ablation catheter from the proximal end of the ablation catheter to the distal portion of the ablation catheter. The distal tip of the thermocouple may be positioned proximally from the fluid reservoir inside the irrigated ablation catheter. The thermocouple may be electrically isolated from the ablation tip of the irrigated ablation catheter.

In another embodiment of the present invention, the distal portion of the elongate body may be configured with a reservoir to receive irrigation fluid or liquid delivered from the proximal end of the elongate body through a fluid lumen to the distal portion of the elongate body. The reservoir may be at least partially enclosed with a cap, shell, housing, or cup shaped metal or metal-alloyed tip, which forms the distal tip of the elongate body. In one variation, the cap, shell, housing, or cup shaped distal tip may be configured with a flat surface. The cap, shell, housing, or cup shaped distal tip may be further configured with a cylindrical surface having a plurality of orifices to allow the fluid to leave the reservoir and exit the catheter. In one example, the flat surface of the cap, shell, housing, or cup shaped tip may have a thickness of less than 0.01 inch.

In another embodiment of the present invention, the ablation catheter comprises an elongate body having an electrode at the distal tip portion. The distal tip may be configured with a flat surface. A fluid reservoir may be located behind the flat portion of the electrode. In one example, the flat portion of the electrode may be less than 0.01 inch, and the reservoir may have a volume of at least 0.00005 cubic inches. Preferably, the outer diameter of the elongate body may be 9 French or less, and a reservoir volume may be at least 0.00006 cubic inches. More preferably, the outer diameter of the elongate body may be 8 French or less and the reservoir volume may be at least 0.00007 cubic inches. The elongate body may further comprises a lumen extending from the proximal portion of the elongate body to the reservoir at the distal portion of the elongate body for supplying a fluid from the proximal portion of the catheter to the reservoir at the distal portion. The distal portion of the elongate body may include a plurality of orifices (e.g., holes) on the circumferential surface of the catheter to allow fluid in the reservoir to exit the elongate body.

In another embodiment of the present invention, optional pull-wires may be embedded or disposed along the length of the elongate catheter body configured for steering the distal section of the catheter. One, two or more wires, threads, thin ropes, etc., may be implemented as pull-wires to steer or articulate various portions of the catheter body. In some variations, the proximal portion of the catheter may be configured to interface with a motorized drive unit or coupler such that a user or operator may direct the movement of the catheter through computers that controls the motors, gears, pulleys, etc. which pull or operate the pull-wires in the catheter to steer or articulate various portions of the catheter. In some other variations, the catheter may be configured to interface with a manually operated drive unit or coupler such that a user or operator may direct the movement of the catheter through various gears or pulleys that pull or operate the pull-wires in the catheter to steer or articulate various portions of the catheter.

In another embodiment, a steerable irrigated ablation catheter may be disposed within a robotically or manually operated steerable sheath catheter such that the ablation catheter may be initially guided toward a target site by the steerable sheath. The steerable sheath may position the irrigated ablation catheter near the target site, and then the steerable irrigated ablation catheter may be further steered or articulated to the target site to perform various procedures.

In another embodiment, a steerable irrigated ablation catheter may be disposed within a robotically or manually operated steerable sheath and guide catheter such that the ablation catheter may be guided toward a target site by the steerable sheath and guide catheter. The sheath and guide catheter may operate in a substantially telescopic manner. That is, the sheath may be steered or articulated to a first location and then the guide may be steered or articulated to a second location which positions the ablation catheter near a target site. At the proximity of the target site, the ablation catheter may be further steered, maneuvered, articulated, or manipulated to perform various operations on the target site or target tissue.

In another variation, the distal tip portion of the ablation catheter may comprise a substantially solid structure having a plurality of channels. The channels may be in fluid communication with the fluid lumen of the catheter. The channels may allow the fluid to enter the structure from the proximal end and exit at a plurality of ports on the peripheries of the structure.

Other and further features and advantages of embodiments of the invention will become apparent from the following detailed description, when read in view of the accompanying figures.

### BRIEF DESCRIPTION OF THE DRAWINGS

The present invention will be readily understood by the following detailed description, taken in conjunction with accompanying drawings, illustrating by way of examples the principles of the invention. The objects and elements in the drawings are not necessarily drawn to scale, proportion, precise orientation or positional relationships; instead, emphasis is focused on illustrating the principles of the invention. The drawings illustrate the design and utility of various embodiments of the present invention, in which like elements are referred to by like reference symbols or numerals. The drawings, however, depict the embodiments of the invention, and should not be taken as limiting its scope. With this understanding, the embodiments of the invention will be described and explained with specificity and detail through the use of the accompanying drawings in which:

FIG. 1A illustrates a cross-sectional view of a distal portion of one embodiment of an ablation catheter having an irrigation lumen. The distal portion of the catheter may be configured with a generally shell-like, thin-walled, or cup-shaped electrode and a reservoir for cooling the electrode.

FIG. 1B illustrates a cross-sectional view of a distal portion of another embodiment of an ablation catheter.

FIG. 1C illustrates a distal section of an ablation catheter.

FIG. 1D illustrates another embodiment of an ablation catheter.

FIG. 2 illustrates one embodiment of an ablation catheter with a built-in pull-wire for steering the distal portion of the catheter. A biasing member (e.g., spring, cantilever, etc.) may be provided to provide counter balance to the pull-wire.

FIG. 3 illustrates another embodiment of an ablation catheter having a pair of pull-wires for steering the distal portion of the catheter.

FIG. 4A illustrates a cross-sectional view of yet another embodiment of an ablation catheter having dual pull-wire construction.

FIG. 4B illustrates one embodiment of an ablation catheter having a manual steering mechanism. The manual steering mechanism may be coupled to a pull-wire embedded or disposed within the body of the elongate catheter for steering the distal portion of the catheter.

FIG. 4C illustrates an embodiment of an ablation catheter having an interface mechanism at the proximal portion of the catheter. The interface is configured for coupling the proximal portion of the catheter to a drive mechanism for controlling the tension of the pull-wires embedded or disposed within the catheter.

FIG. 4D illustrates one embodiment of a combination of an ablation catheter and a manually operated sheath.

FIG. 4D1 illustrates one embodiment of a combination of a steerable ablation catheter and a manually operated sheath.

FIG. 4E illustrates one embodiment of a combination of an ablation catheter and a robotically operated sheath.

FIG. 4E1 illustrates one embodiment of a combination of a steerable ablation catheter and a robotically operated sheath.

FIG. 4F illustrates one embodiment of a combination of an ablation catheter and a manually operated sheath and guide system.

FIG. 4G illustrates one embodiment of a combination of an ablation catheter and a robotically operated sheath and guide system.

FIG. 5 illustrates a cross-sectional view of another embodiment of an irrigated ablation catheter. In this embodiment, the inner irrigation tube protrudes into the reservoir located at the distal tip portion of the ablation catheter.

FIG. 6A illustrates a cross-sectional view of yet another embodiment of an irrigated ablation catheter. In this embodiment, the distal tip may be configured with a rounded profile.

FIG. 6B illustrates a cross-sectional view of an embodiment of a round-tip irrigated ablation catheter, wherein the inner irrigation tubing protrudes into the reservoir.

FIG. 6C illustrates a cross-sectional view of another embodiment of a round-tip irrigated ablation catheter. In this embodiment, the distal tip may be configured with a solid metallic block, instead of a substantially thin wall, under the substantially hemispherical surface of the structure.

FIG. 7 illustrates a cross-sectional view of another embodiment of an irrigated ablation catheter having the inner irrigation tubing protruding into the distal reservoir.

FIG. 8A illustrates a cross-sectional view of yet another embodiment of an irrigated ablation catheter. In this configuration, the distal electrode portion of the ablation catheter comprises a solid structure having a substantially centrally located lumen with a plurality of channels extending radially to ports located on the cylindrical surface of the distal tip electrode.

FIG. 8B illustrates another view of the electrode shown in FIG. 8A.

FIG. 9A illustrates a cross sectional view of another embodiment of an irrigated ablation catheter.

FIG. 9B illustrates a close-up cross sectional view of the thermocouple of Detailed View A-A of FIG. 9A.

FIG. 9C illustrates a close-up cross sectional view of the thermocouple in accordance with one embodiment of the present invention.

FIG. 9D through FIG. 9F illustrates various positions of a temperature sensing element within a thermocouple in accordance with embodiments of the present invention.

FIG. 9G through FIG. 9I illustrate various locations or positions of the thermocouple as it may be disposed in the irrigated ablation catheter in accordance with embodiments of the present invention.

### DETAILED DESCRIPTION OF ILLUSTRATED EMBODIMENTS

Reference will now be made in detail to the preferred embodiments of the present invention, examples of which are illustrated in the accompanying drawings. While the invention will be described in conjunction with the preferred embodiments, it will be understood that they are not intended to limit the scope of the invention to these embodiments. On the contrary, the invention is intended to cover alternatives, modifications, and equivalents that may be included within the spirit and scope of the invention. Furthermore, in the following detailed description of the present invention, numerous specific details are set forth in to order to provide a thorough understanding of the present invention. However, it will be readily apparent to one of ordinary skilled in the art that the present invention may be practiced without these specific details.

It should be understood that embodiments of the present invention may be applied in combination with various catheters, tubing introducers, access sheath or other medical deployment devices for implementation within a subject's body. It must also be noted that, as used in this specification and the appended claims, the singular forms “a,” “an” and “the” include plural referents unless the context clearly dictates otherwise. Thus, for example, the term “a tube” is intended to mean a single tube or a combination of tubes, “a fluid” is intended to mean one or more fluids, or a mixture thereof.

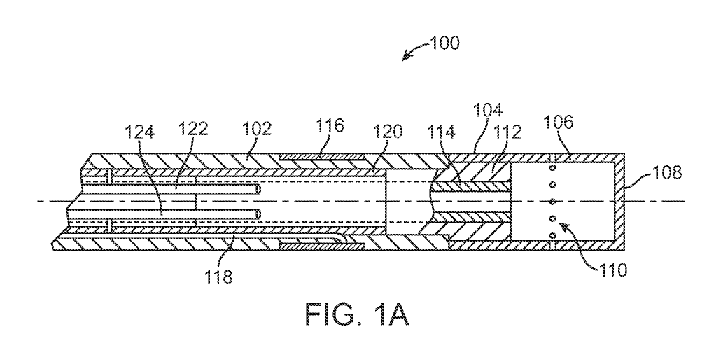

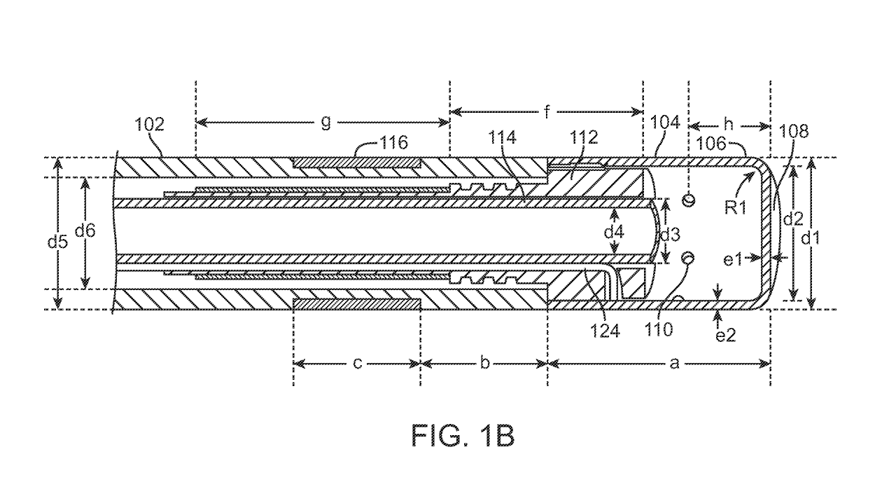

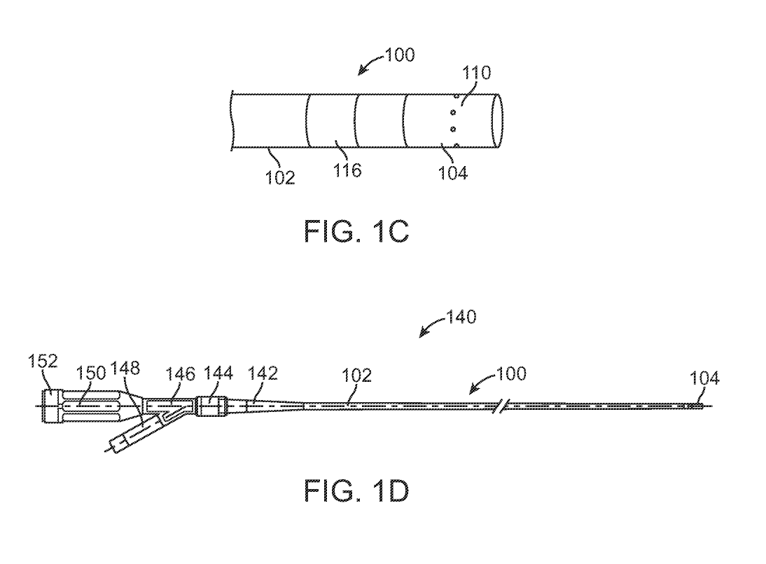

FIG. 1A illustrates one embodiment of an irrigated ablation catheter (100) that may be configured for ablating tissue structures in minimally invasive procedures. The irrigated ablation catheter (100) may include an elongate body (102) and an ablation member (104). The elongate body (102) may be a tubular member having substantial flexibility. The distal portion of the elongate body (102) may be more flexible than the proximal portion. For example, the distal portion of the elongate body may be constructed from a material having a durometer rating or stiffness of about 40D, while the proximal portion of the elongate body may be constructed from a material having a durometer rating or stiffness of about 70D. The ablation member may be fabricated from a substantially conductive material. In one embodiment, the ablation may be made of stainless steel. In another embodiment, the ablation tip member may be made of platinum or a platinum alloy, such as platinum and iridium. For a platinum alloy, the composition may be about 90 percent platinum and about 10 percent iridium. The ablation member (104) may be coupled to the elongate body (102) by any conventional means, such as thermally fusing the proximal end of the ablation member (104) to the distal end of the elongate body (102). Pebax is one example of a thermal plastic that may be used to thermally fuse the ablation member (104) to the elongate body (102). The ablation member (104) includes a body (106) that may be substantially cylindrical and a distal or tip surface (108) that may be substantially flat. In other embodiments, the body (106) may be substantially rectangular or any suitable shape and size. Similarly, the elongate body (102) may be substantially cylindrical, substantially rectangular, or any suitable shape and size. The ablation catheter may be used in combination with a steerable sheath catheter. In addition to being substantially flat, the tip surface (108) may be substantially round or substantially hemispherical. The body (106) of the ablation member (104) may include a plurality of ports, openings, or holes (110) for dispensing cooling fluid, such as biologically compatible saline solution, out of the ablation member (104). In other embodiments, the plurality of ports or openings (110) may be located on the distal or tip surface (108) instead of on the body (106). In further embodiments, the plurality of ports or openings (110) may be located on both the body (106) and the tip surface (108).

Still referring to FIG. 1A, the irrigated ablation catheter (100) may include a support member (112) and an inner tube (114). The support member (112) may be an insert, such as a stainless steel insert. The inner tube (114) may include a lumen. The tube (114) may be configured to carry cooling fluid from the proximal portion of the catheter (100) to the ablation member or tip structure (104). The cooling fluid cools the ablation member or tip structure (104) from the inside as the tip structure may heat up during ablation operations. In addition, the cooling fluid may be dispensed through the ports or opening (110) of the tip structure (104), such that the tip structure may be cooled from the outside by having the cooling fluid flowing over the exterior surface of the tip structure (104). Furthermore, the surface of the tissue that is being ablated may be also cooled by the cooling fluid, such that a deeper and more uniform lesion may be formed by the irrigated ablation catheter (100). In other words, cooling of the tissue structure provided by the irrigated ablation catheter prevents surface charring of the tissue such that deeper and more uniform lesion may be formed in underlying tissue that is being ablated.

The irrigated ablation catheter (100) may further include a ring electrode (116) and a conductor wire (118) for electrically coupling the ring electrode (116) to a power, control, and monitoring system, such as an RF generator having control and monitoring capabilities. The ring electrode (116) may be used in either mono-polar or bi-polar sensing mode. The ring electrode (116) may be used in bi-polar sensing along with the tip structure (104) to determine condition of the tissue during or after tissue ablation. The conductor (118) may be supported and insulated. In this example, the conductor (118) may be supported and insulated by a tube member (120).

FIG. 1B illustrates another embodiment of an irrigated ablation catheter (100). The catheter (100) includes an elongate body (102) and an ablation tip member (104). In order to illustrate the relative sizes of the components of the irrigated ablation catheter (100), the following dimensions are provided for illustrative purposes:

(a) may be in the range of about 1.5 mm to about 4.5 mm; in some embodiments (a) may be about 4 mm.

(b) may be about 0.5 mm to about 3 mm.

(c) may be in the range of about 1.0 mm to about 2.5 mm; in some embodiments (c) may be about 2 mm.

(d1) may be about 0.092 in or about 7 French.

(d2) may be about 0.074 in.

(d3) may be about 0.040 in.

(d4) may be about 0.026 in.

(d5) may be about 0.092 in.

(d6) may be about 0.072 in.

(e1) may be in the range of about 0.005 in to about 0.015 in.; or in the range of about 0.006 in to about 0.010 in.; or in the range of about 0.007 in to about 0.009 in; or about 0.009 in.

(e2) may be in the range of about 0.005 in to about 0.015 in.; or in the range of about 0.006 in to about 0.010 in; or in the range of about 0.007 in to about 0.009 in; or about 0.009 in.

The flat area of the tip surface (108) may be about 0.001 in² to about 0.015 in²; or may be about 0.002 in² to about 0.010 in²; or about 0.002 in² to about 0.005 in².

(f) may be about 4 mm.

(g) may be in the range of about 4 mm to about 10 mm; or about 6 mm.

(h) may be about 1.25 mm.

Diameter of port or opening (110) may be in the range of about 0.006 in to about 0.018 in.; or about 0.11 in.

There may be about 5 to about 10 ports or opening (110); in one embodiment there may 7 ports or openings (110); 11 or more ports (110) may also be implemented.

(R1) may be 0.01 in.

FIG. 1C illustrates one embodiment of an irrigated ablation catheter (100). The catheter (100) includes an elongate member (102), ablation tip member (104), and an electrode ring (116). The ablation tip member (104) may include a plurality of ports or opening (110) for dispensing cooling fluid.

FIG. 1D illustrates one embodiment of an irrigated ablation catheter assembly (140). As illustrated in FIG. 1D, the assembly may include an ablation tip structure (104), an elongate body (102), a support member (142), coupling member (144), a Y-joint (146), a fluid coupling (148), an electrical coupling (150), and a plug member (152). The Y-joint (146) provides electrical and fluid supply and isolation to the irrigated ablation catheter (100). Irrigation supply may be coupled to the fluid coupling (148) and power supply for ablation may be coupled to the plug member (152).

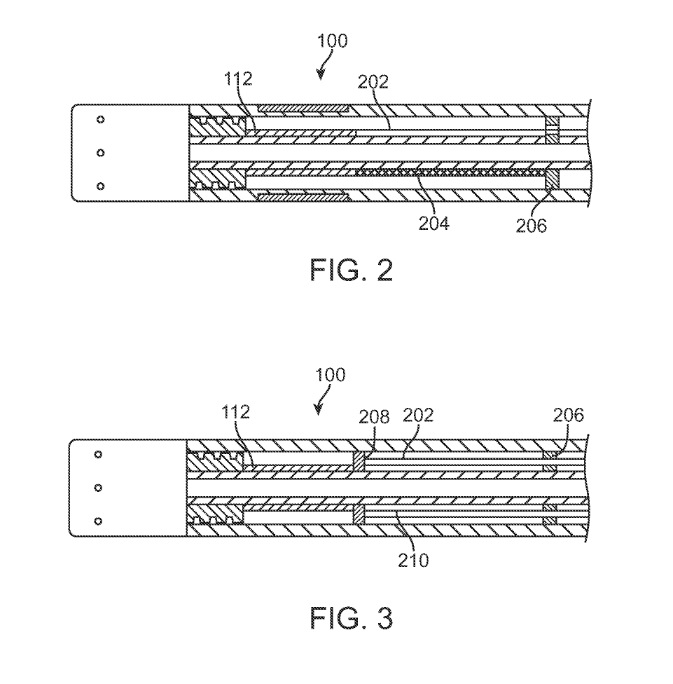

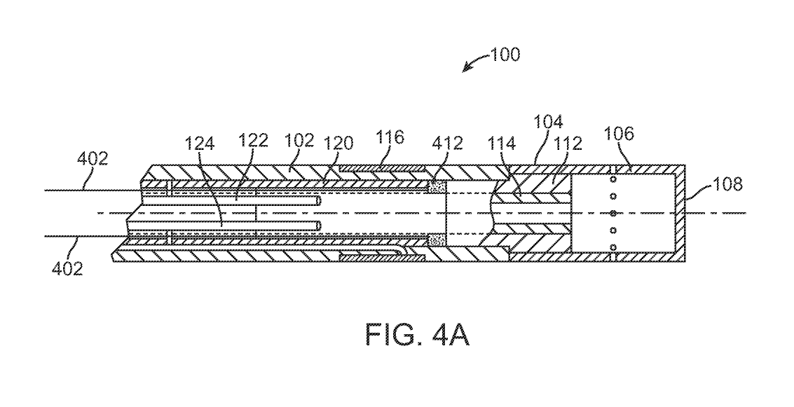

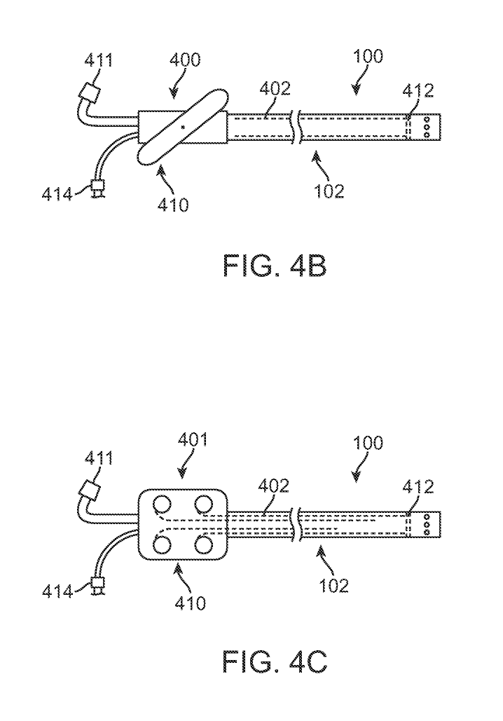

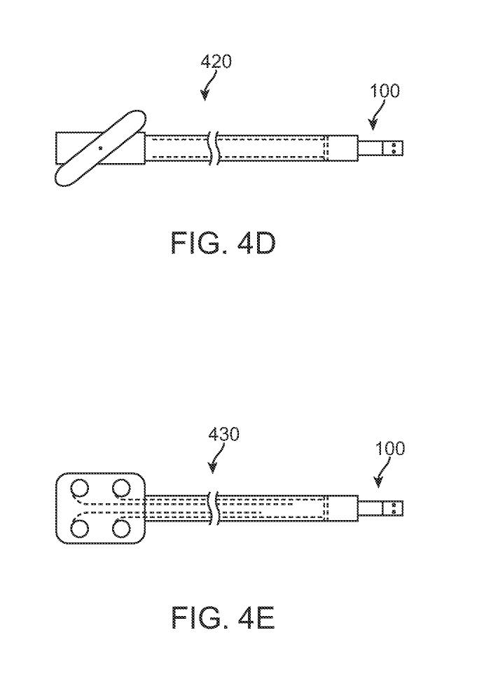

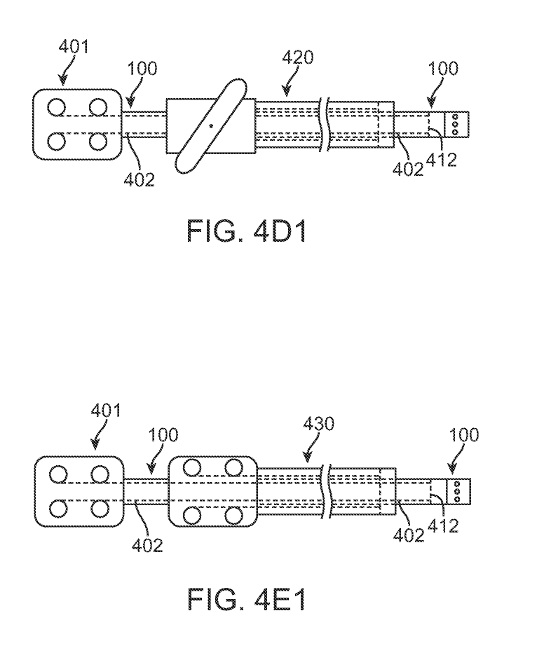

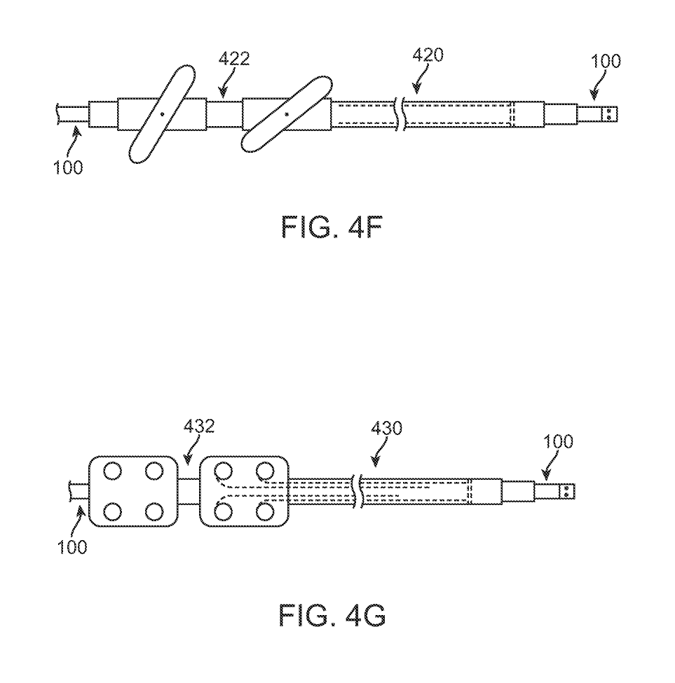

FIG. 2 illustrates another embodiment of an irrigated ablation catheter (100) in which a control or pull wire (202) and a spring rod (204) are incorporated to allow deflection or steering control of the irrigated ablation catheter (100). The control wire (202) and the spring rod (204) may be secured to the support member (112) near the distal portion of the catheter (100) at one end. While at the other end, the control wire (202) may be threaded through a support ring (206) and then operatively coupled to a steering system, and the spring rod (204) may be secured to the support ring (206). The steering system may be either a manually controlled steering system, similar to the system (400) as illustrated in FIG. 4B, or a robotically controlled steering system, similar to the system (401) as illustrated in FIG. 4C. The catheter (100) may be deflected or steered by activating or tensioning the control wire (202). The catheter (100) may return to its substantially neutral position or orientation when the tension on the control wire (202) is released and the spring rod (204) provides the necessary spring force to deflect or steer the catheter to restore its substantially neutral position.

FIG. 3 illustrates a catheter (100) having two control wires (202). The control wires (202) may be secured to the support member (112) or the control wires (202) may be secured to a control ring (208). The control ring (208) may be secured near the distal portion of the catheter (100). For example, the control ring (208) may be secured near the support member (112). The control wires (202) may be slidably coupled to tubings (210) to protect the control wires (202). The tubings (210) may be secured to or supported by the control ring (208) and/or support ring (206).

FIG. 4A illustrates another implementation of control wires to deflect or steer the catheter (100). As illustrated in FIG. 4A, two control wires (402) are secured to a control ring (412) position near the distal end of the elongate body (102) or near the proximal end of the ablation tip member (104).

Referring to FIGS. 4B and 4C, irrigation may be provided to the irrigated ablation catheter (100) by way of the fluid connector (411), and ablation energy may be provided to the irrigated ablation catheter (100) by way of the power connector (414). Although not shown in FIG. 4B or FIG. 4C, the fluid connector (411) may be coupled to an irrigation system to supply suitable amount of cooling fluid to the catheter (100). For example, the irrigation system may supply cooling fluid at a flow rate in the range of about 2 milli-liters per minute (ml/min) to about 30 milli-liters per minute (ml/min) at a pressure in the range of about 2 pounds per square inch (psi) to about 30 pounds per square inch (psi). In some implementations, the pressure may be in the range of about 5 pounds per square inch (psi) to about 30 pounds per square inch (psi). In other implementations, other pressure ranges may also be preferable. In some implementations, the cooling fluid flow rate may be about 17 ml/min at about a pressure of about 12 psi. In one implementation, the cooling fluid flow rate may be at about 30 ml/min at a pressure of about 24 psi. In another implementation, the cooling fluid flow rate may be at about 30 ml/min at a pressure of about 35 psi. Although not shown in FIG. 4B or FIG. 4C, the power connector (214) may be coupled to an energy supply system, such as an RF generator, control, and monitoring system, to supply suitable amount of energy to the ablation catheter (100) and tip structure (104) to ablate various tissue structures. A conductor (122) electrically couples the power connector (414) with the ablation tip member (104). An energy supply system may provide up to about 30 watts of power for ablation. In some implementations, the energy supply system may provide over 30 watts of power for ablation. In some ablation procedures, about 70 watts of power may be used for performing ablation. However, a typical ablation procedure may use about 30 watts of power to ablate tissue to form lesion on the underlying tissue structure. A thermocouple (124) monitors the temperature of the tip structure (104), such that the surgeon who is performing the ablation procedure may vary the amount of irrigation and/or power of the irrigated ablation catheter (100) so that an appropriate lesion may be formed on the tissue that is being ablated. The irrigated ablation catheter (100) is designed to be a flexible system, such that it may be coupled to various state-of-the-art irrigation supply system and energy supply system such that the necessary amount of irrigation and energy could be used for ablating and cooling for tissue ablation.

Still referring to FIG. 4B and FIG. 4C, to steer the irrigated ablation catheter (100), control wires or pull wires (402) may be coupled to various points or locations along the elongate body (102) or to deflect the elongate body in various manners for steering or navigating the catheter (100) to various anatomical structures through various natural pathways in the anatomy of a patient. FIG. 4B illustrates one embodiment of a manually controlled steering system (400) for steering the ablation catheter (100). Pull wires (402) may be coupled to various points or locations of the elongate body (102), and the tip (104) may be steered as the pull wires (402) are manipulated by way of a control handle (410). In this example, the pull wires (402) may be anchored to a control ring (412) that may be is located near the distal portion of the elongate body (102). As may be appreciated, the control wires (402) may be anchored in other manners and a control ring may not be used at all for anchoring the control wires (402). For example, the control wires (402) may be anchored on any points or locations of the elongate body; such as being incorporated in the tubing structure, wire braiding, or mesh weaving of the elongate body (102) or to the tip insert (112). As the control handle (410) is turned one way or another the control wires (402) may be tensioned or relaxed, such that the elongate body (102) may be deflected in one direction or another. Similarly, FIG. 4C illustrates one embodiment of a robotically controlled steering system (401) for steering the ablation catheter (100). Pull wires (402) may be coupled to various points or locations of the elongate body (102), and the tip (104) may be steered as the pull wires (402) are manipulated robotically, e.g., pulleys, gears, motors, etc., by way of the robotic control system (410). The robotic control steering system (410) may be coupled to a drive system (not shown) having drive motors controlled by a computer to operate various gears or pulleys in the robotic system (410) to operate the control wires (402). The operations of the control wires (402) steer or articulate various portions of the elongate body (102). In this example, some of the pull wires (402) may be anchored to a control ring (112) that may be located near the distal portion of the elongate body (102) while some of the control wires (402) may be anchored along the elongate body (102). The control wires (402) may be anchored on any points or locations of the elongate body; such as being incorporated in the tubing structure, wire braiding, or mesh weaving of the elongate body (102) or the tip insert (112). As the pulleys, gears, etc. of the robotically controlled steering system (410) is turned one way or another, the control wires (402) may be tensioned or relaxed, such that the elongate body (102) may be deflected in one direction or another. Although in this example four control wires (402) are illustrated, more or fewer control wires (402) may be used. The control wires (402) may be anchored to a control ring (412) or any points or locations along the elongate body (102).

Referring back to FIG. 4A of the irrigated ablation catheter (100), a control ring (412) may be positioned near the proximal end of the irrigated ablation tip structure (104) for which control wires (illustrated in FIG. 4B and FIG. 4C) may be anchored for deflecting the elongate body (102) and steering the irrigated ablation tip structure (104). Although the control ring (412) is shown as located near the proximal end of the tip structure (104), the control ring, if it is used, may be located at any location along the length of the elongate body (102) of the catheter (100).

In another embodiment, a non-steerable irrigated ablation catheter may be disposed within a manually operated steerable sheath catheter (420) or a robotically operated steerable sheath (430) such that the non-steerable ablation catheter may be guided toward a target site by the steerable sheath as illustrated in FIG. 4D and FIG. 4E. The steerable sheath (420 or 430) may position the irrigated ablation catheter near the target site, and then the steerable sheath (420 or 430) may then guide the non-steerable irrigated ablation catheter to perform various procedures on a target site or target tissue. Similarly, in another embodiment a steerable irrigated ablation catheter (100) may be disposed within a manually operated steerable sheath catheter (420) or a robotically operated steerable sheath catheter (430) such that the ablation catheter (100) may be initially guided toward a target site by the steerable sheath (420 or 430) as illustrated in FIG. 4D1 and FIG. 4E1. The steerable sheath (420 or 430) may position the irrigated ablation catheter (100) near the target site, and then the steerable irrigated ablation catheter (100) may be further steered or articulated to the target site to perform various procedures.

In another embodiment, a steerable irrigated ablation catheter (100) may be disposed within a manually operated steerable sheath and guide catheter (420 and 422) or a robotically operated sheath and guide catheter (430 and 432) such that the ablation catheter may be guided toward a target site by the steerable sheath and guide catheter as illustrated in FIG. 4F and FIG. 4G. The sheath and guide catheter may operate in a substantially telescopic manner. That is, the sheath may be steered or articulated to a first location and then the guide may be steered or articulated to a second location which positions the ablation catheter (100) near a target site. At the proximity of the target site, the ablation catheter (100) may be further steered, maneuvered, articulated, or manipulated to perform various operations on the target site or target tissue. Similarly, in another embodiment, a non-steerable irrigated ablation catheter may be disposed within a manually operated steerable sheath and guide catheter (420 and 422) or a robotically operated sheath and guide catheter (430 and 432) such that the ablation catheter may be guided toward a target site by the steerable sheath and guide catheter. The sheath and guide catheter may operate in a substantially telescopic manner. That is, the sheath may be steered or articulated to a first location and then the guide may be steered or articulated to a second location which positions the ablation catheter near a target site. At the proximity of the target site, the ablation catheter may be further steered, maneuvered, articulated, or manipulated by the guide catheter to perform various operations on the target site or target tissue.

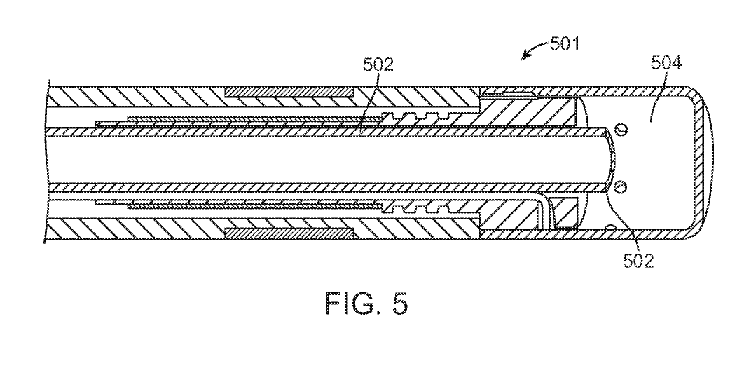

Referring to FIG. 5, another embodiment or variation of an irrigated ablation catheter (501) is illustrated. In this variation, the distal end (502) of the inner irrigation tube (503) protrudes substantially into the reservoir (504) located near the distal portion of the catheter.

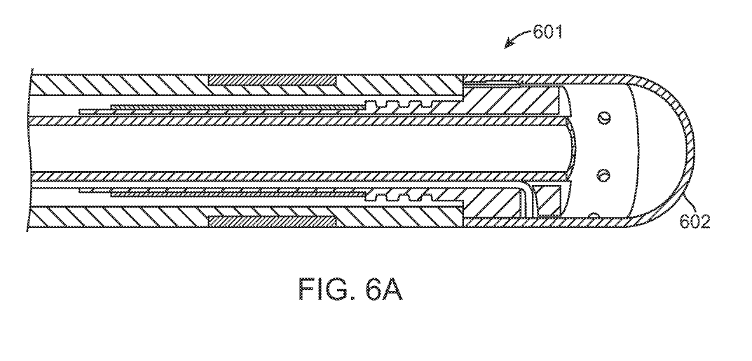

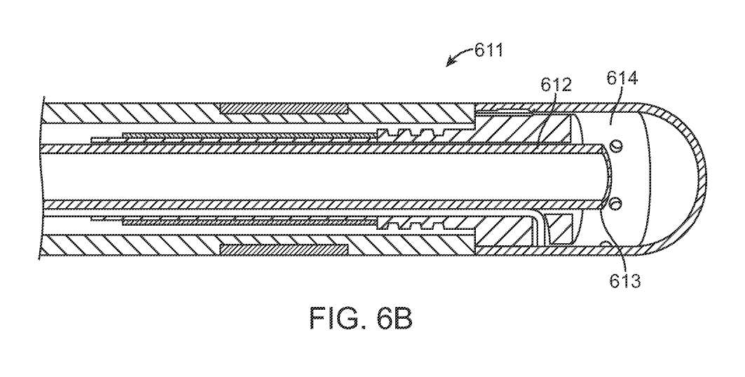

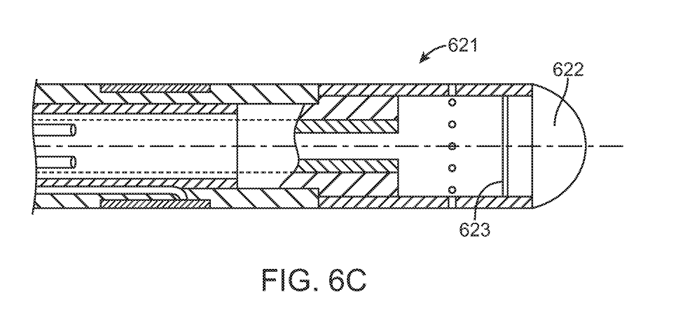

Referring to FIG. 6A another embodiment or variation of an irrigated ablation catheter (601) is shown. In this variation, the distal tip (602) may be configured with a substantially rounded tip. The rounded tip may be configured to be a substantially hemispherical shape.

FIG. 6B illustrates a variation of a round tip irrigated ablation catheter (611) where the distal end (613) of the inner irrigation tube (612) protrudes substantially into the reservoir (614).

FIG. 6C illustrates yet another embodiment or variation of a round tip irrigated ablation catheter (621). In this variation, the distal tip electrode (622) is configured with a substantially solid structure (622) with a substantially hemispherical outer surface. In other words, in some embodiments an ablation electrode may be a substantially thin wall structure, while in some other embodiments, an ablation electrode may be a substantially solid structure. The proximal surface (623) of the substantially solid structure (622) may define or provide the distal boundary of an irrigation reservoir for the irrigated ablation catheter (621).

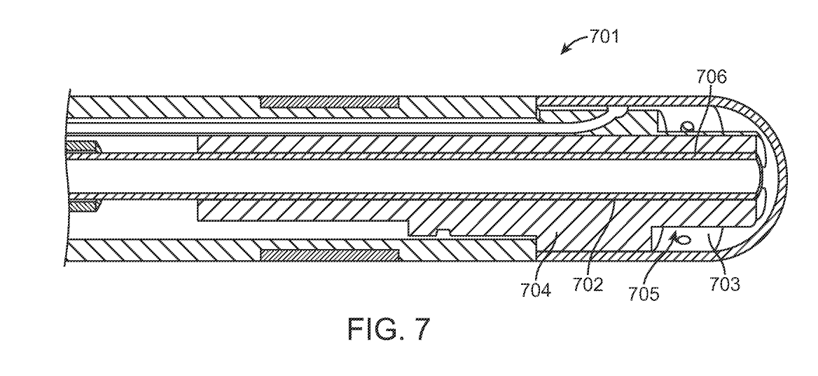

Referring to FIG. 7, another variation of an irrigated ablation catheter (701) having an inner irrigation tube (702) protruding substantially into the distal irrigation reservoir (703) is shown. In this variation, the inner metallic support ring (704) may also protrude substantially distally (705) into the irrigation reservoir to support the distal section (706) of the irrigation tube (702).

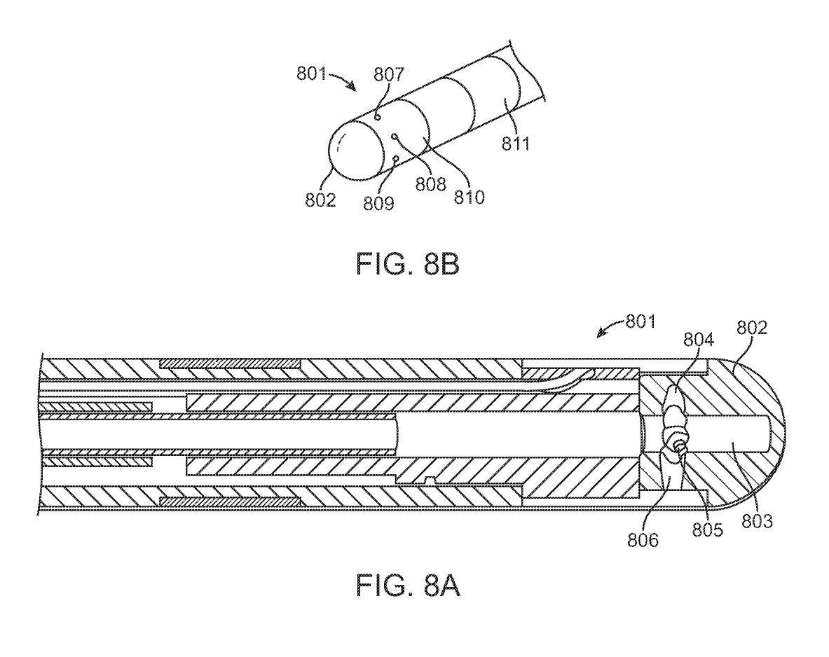

FIG. 8A illustrates yet another variation of an irrigated ablation catheter (801). In this variation, the ablation member (802) of the ablation catheter comprises a substantially solid structure having a centrally located lumen (803) with a plurality of channels (804, 805, 806) extending substantially radially to a plurality of ports (807, 808, 809) located on the substantially cylindrical surface (810) of the distal tip electrode (802), shown in FIG. 8B. In this example, an optional secondary electrode (811) may also be provided. The secondary electrode (811) may be configured for bipolar electric activity sensing when used along with the primary distal electrode (802). Similarly, in another variation, an ablation member of the ablation catheter may comprise of a substantially thin-shell structure having a substantially centrally located lumen distal tip electrode and an optional secondary electrode. The secondary electrode may be configured for bipolar electric activity sensing when used along with the primary distal electrode.

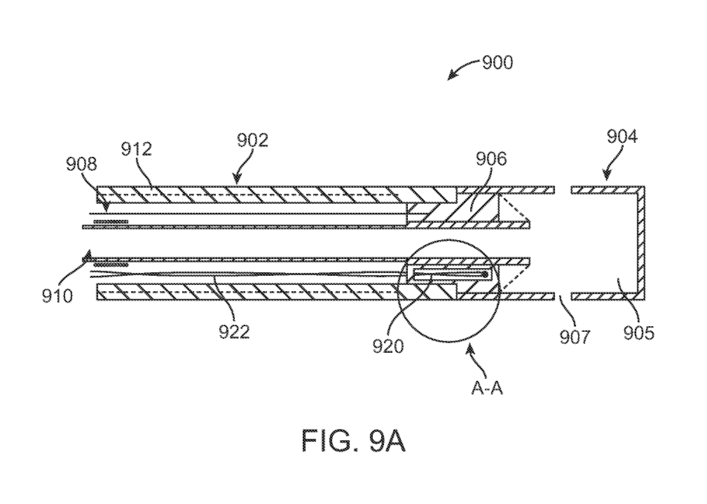

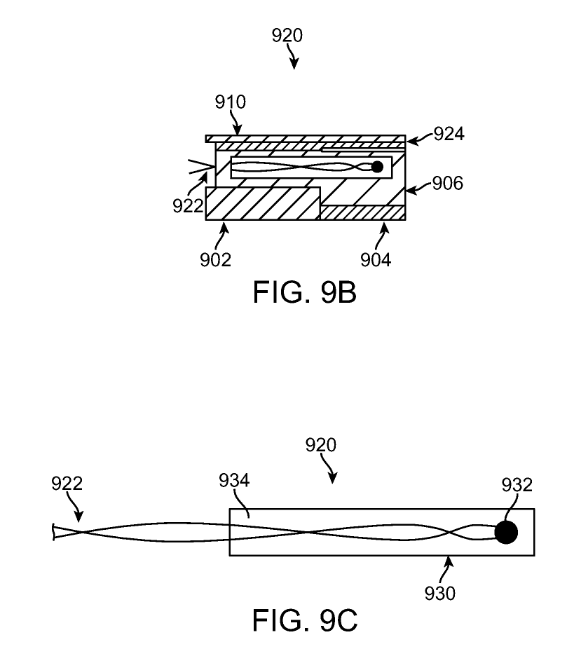

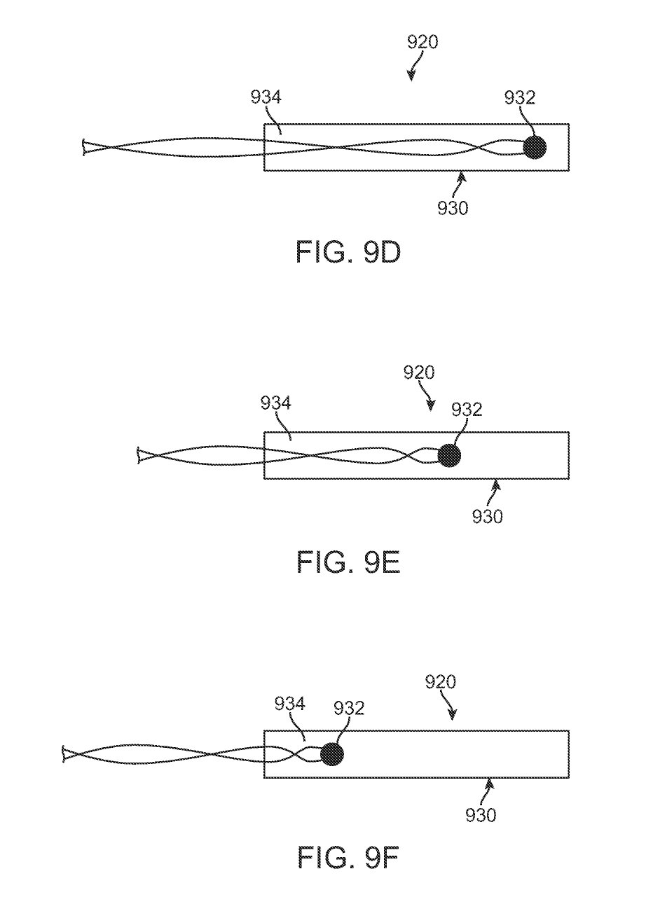

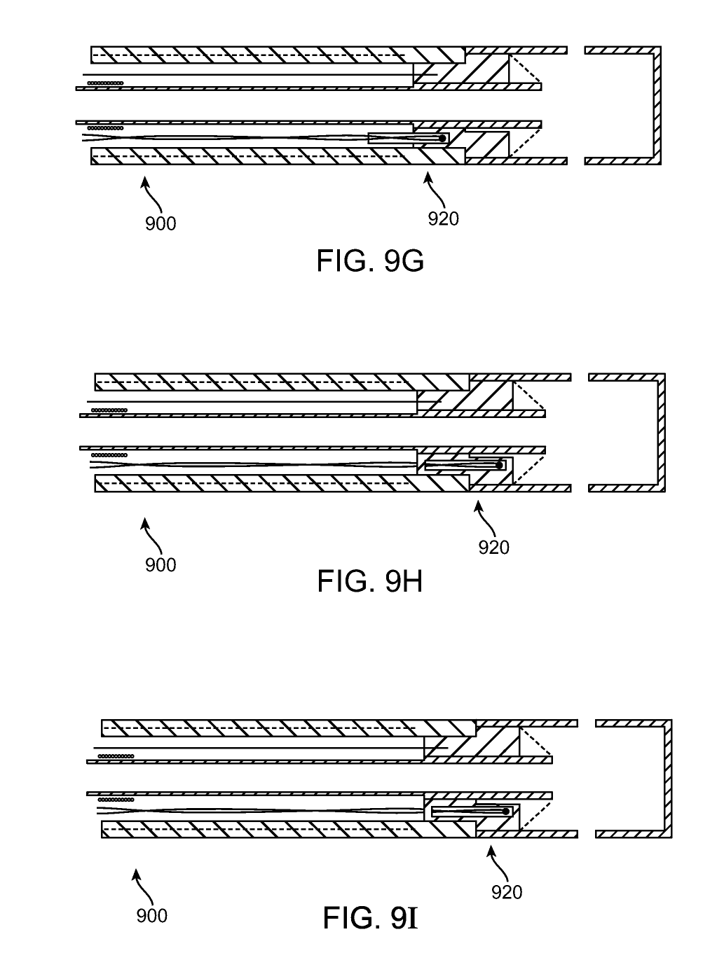

FIG. 9A illustrates a cross sectional view another embodiment of an irrigated ablation catheter. As illustrated in FIG. 9A, the irrigated ablation catheter (900) includes an elongate body (902), an ablation member (904), an insert (906), a safety wire or tether (908), an irrigation tube (910), a thermocouple (920), and thermocouple wires (922). The ablation member (904) may include a cavity or reservoir (905). The reservoir (905) may be in fluid communication with the irrigation tube (910) such that irrigation fluid may be delivered from the proximal portion of the irrigated ablation catheter to the distal portion of the irrigated ablation catheter into the reservoir (905). Irrigation fluid may be used to cool the distal portion of the ablation member (904) during ablation procedures. The ablation member (904) may also include ports (907) on the side wall of the ablation member. The ports (907) allow irrigation fluid to be dispensed out of the reservoir (905) such that irrigation fluid may cool the exterior surface of the ablation catheter. In other words, the ablation member (904) may be cool internally by the irrigation fluid in the reservoir (905) as well as externally by dispensing irrigation fluid through the ports (907) to the exterior surface of the ablation member (905). The cooled surface of the ablation member (905) may in turn cool the tissue that is being ablated by the ablation catheter. Furthermore, as irrigation fluid is dispensed through the ports (907) of the ablation member (907), the irrigation fluid may also provide cooling to the ablated tissue.

Still referring to FIG. 9A, the thermocouple (920) may be disposed proximally to the reservoir (905) to measure as well as monitor the temperature of the ablation member (904). Placing the thermocouple (920) proximal to the reservoir (905) may be the optimal location to measure the temperature of the ablation member (904). The area or region proximal to the reservoir (905) may provide a more accurate or useful temperature measurement for the ablation member (905) for monitoring or controlling the temperature of the ablation member (905) during ablation procedures to avoid overheating the tissue that is being ablated. In particular, using the ablation catheter or the ablation member to cool the tissue during ablation prevents charring the surface of the tissue structure such that deeper and more uniform lesion may be achieved.

FIG. 9B illustrates a close-up cross sectional view of the thermocouple of Detailed View A-A in FIG. 9A. As illustrated in FIG. 9B, the thermocouple (920) may be disposed within an insert (906). The insert (906) made be fabricated from a thermal conductive material such as stainless steel or a substantially thin layer of a material, e.g., a polyimide material. The insert (906) may be secured to the elongate body (902) by various means. For example, the insert (906) may be adhesively bonded to the elongate body (902) by an adhesive material (924) such as thermally conductive epoxy. Additional details of the thermocouple (920) are illustrated in FIG. 9C.

FIG. 9C illustrates a close-up view of the thermocouple in accordance with one embodiment of the present invention. As illustrated in FIG. 9C, the thermocouple (920) may include a covering (930). The covering (930) provides an electrically isolating barrier to the thermocouple (920) to ensure accurate temperature measurement in or near an electrically conductive or noisy environment due to it proximity to the ablation member (905). The covering (930) may be a thin-walled polyimide tube. The temperature sensing element (932) of the thermocouple (920) may be potted inside the covering (930) with a thermally conductive material such as a thermally conductive epoxy (934). The thermally conductive material may also be electrically insulating. The length or thickness of the covering (930) may be varied to achieve the desired temperature response or temperature measurement or sensing characteristics. In addition, the location of the thermocouple (920) may be varied axially to achieve the desired temperature response or temperature sensing characteristics. In some embodiments, the thermocouple (920) may be located at about 0.5 mm to about 1.5 mm from the distal end of the insert (906). In some embodiments, the thermocouple (920) may be located or positioned at about 1 mm from the distal end of the insert (906). Similarly, the position or location of the thermocouple (920) may be varied radially.

In addition, the position of the temperature sensing element (932) within the covering (930) may be varied to obtain the desired temperature sensing response or characteristics. For example, as illustrated in FIG. 9D through FIG. 9F, the temperature sensing element (932) may be varied axially within the covering (930) or the potting material (934) obtain the desired temperature sensing response or temperature sensing characteristics. For example, the axial location of the thermocouple sensing element (932) may be varied by moving the thermocouple sensing element (932) to different axial positions along the casing or covering (930) or potting material (934) along the length of the covering (930) to different axial locations within the electrically insulating material (934) and the covering (930). For example, in some embodiments, the temperature sensing element (932) may be located or positioned at about 0.1 mm to about 4 mm from the distal tip or distal end of the thermocouple (920). In some embodiments, the temperature sensing element (932) may be located or positioned at about 0.7 mm from the distal tip or distal end of the thermocouple (920). By varying the location of the thermocouple temperature sensing element (932), the thermal response or temperature sensing characteristics may vary. In one embodiment, the location of the thermocouple temperature sensing element (932) may be selected or configured to substantially match the thermal response of standard temperature sensing catheters. In other embodiments, the position of the thermocouple sensing element (932) may be varied to achieve different thermal responses or sensing characteristics. Similarly, the temperature sensing element (932) may also be varied radially.

The thermocouple wires (922) may be coupled or connected to a thermocouple control system (not shown) at or near the proximal portion of the ablation catheter (900). FIG. 9G through FIG. 9I illustrate various locations or positions of the thermocouple (920) as it may be disposed in the irrigated ablation catheter for optimal temperature sensing. As illustrated, the axial position of the thermocouple (920) may be varied for placement at a location that may provide optimal temperature sensing in the catheter. In addition, although not illustrated the radial location of the thermocouple (920) may also be varied to optimize the temperature sensing characteristics. For example, in one embodiment, the thermocouple (920) may not make contact with the elongate body (902) or the irrigation tube (910) as illustrated in FIG. 9G and FIG. 9I. In another embodiment, as illustrated in FIG. 9H, the thermocouple (920) may contact with the elongate body (902). The axial location of the thermocouple may be varied by moving the thermocouple assembly (920) to different axial positions along the length of the catheter. By varying the location of the thermocouple, the thermal response or temperature sensing characteristics may be varied. In one embodiment the location of the thermocouple may be selected or configured to substantially match the thermal response or sensing characteristics of standard temperature sensing catheters. In other embodiments, the position of the thermocouple may be configured to achieve different thermal responses or sensing characteristics. Similarly, the location of the thermocouple (920) may also be varied radially within the ablation catheter.

As have been discussed in this disclosure, irrigated ablation catheters in accordance with embodiments of the present invention may include various components (e.g., pull-wires, etc.) and mechanisms (e.g., pulleys, gears, etc.) to allow manual or robotic steering or articulation of various portions of the irrigated ablation catheter. Embodiments of the present invention may also include none self-steering or none self-articulating, neither manually nor robotically, irrigated ablation catheters. Such non self-steerable or non self-articulating irrigated ablation catheters may be coupled, installed, mounted, or incorporated into or in combination with steerable systems such as the Artisan™ Control Catheter system from Hansen Medical in Mountain View, Calif., U.S.A. As such, the non self-steerable or non self-articulating irrigated ablation catheters may be steered or articulated by a sheath and guide system or an outer guide and inner guide system. Alternatively, the non self-steerable or non self-articulating irrigated ablation catheters may be steered or articulated with just one steerable guide.

Multiple embodiments and variations of the various aspects of the invention have been disclosed and described herein. Many combinations and permutations of the disclosed system may be useful in minimally invasive medical intervention and diagnostic procedures, and the system may be configured to support various flexible robotic instruments. One of ordinary skill in the art having the benefit of this disclosure would appreciate that the foregoing illustrated and described embodiments of the invention may be modified or altered, and it should be understood that the invention generally, as well as the specific embodiments described herein, are not limited to the particular forms or methods disclosed, but also cover all modifications, equivalents and alternatives. Further, the various features and aspects of the illustrated embodiments may be incorporated into other embodiments, even if not so described herein, as will be apparent to those ordinary skilled in the art having the benefit of this disclosure. Although particular embodiments of the present invention have been shown and described, it should be understood that the above discussion is not intended to limit the present invention to these embodiments. It will be obvious to those skilled in the art that various changes and modifications may be made without departing from the spirit and scope of the present invention. Thus, the present invention is intended to cover alternatives, modifications, and equivalents that may fall within the spirit and scope of the present invention as defined by the claims.

What is claimed is:

1. An apparatus for tissue ablation, comprising:
an elongate flexible member having a proximal end and a distal end;
an irrigation lumen disposed between the proximal end and the distal end of the elongate flexible member;
an ablation member coupled to the distal end of the elongate flexible member, wherein the ablation member comprises a shell having a side wall and a distal wall, the side wall and distal wall defining an irrigation reservoir, the irrigation reservoir being in fluid communication with the irrigation lumen, the side wall includes a plurality of ports, the plurality of ports being in fluid communication with the irrigation reservoir; and
a thermocouple disposed from the proximal end of the elongate flexible member to a distal portion of the elongate member, wherein a distal tip of the thermocouple being positioned proximal of the irrigation reservoir and the thermocouple being electrically isolated from the ablation member.

2. The apparatus of claim 1, further comprising a plurality of pull-wires slideably disposed within the elongate flexible member, wherein the plurality of pull-wires extend from the proximal end to the distal portion of the elongate flexible member, and the distal ends of the plurality of pull-wires being coupled to the distal portion of the elongate flexible member for steering the distal portion of the elongate flexible member.

3. The apparatus of claim 2, further comprising a mechanical coupler attached to the proximal end of the elongate flexible member, wherein the mechanical coupler includes a plurality of rotatable members coupled to the plurality of pull-wires, the rotatable members are configured to engage one or more electrical motors.

4. The apparatus of claim 2, further comprising a plug coupling the ablation member to the distal end of the elongate flexible member, wherein the plug includes a channel providing fluid communication between the irrigation lumen and the irrigation reservoir, the thermocouple being positioned within the plug and electrically isolated from the plug.

5. The apparatus of claim 4, wherein the plug is a metallic plug.

6. The apparatus of claim 4, further comprising a wire extending from the proximal end to the distal portion of the flexible elongate flexible member, the distal end of the wire being connected to the ablation member.

7. The apparatus of claim 1, wherein the side wall of the shell being substantially cylindrical, and the distal wall of the shell being substantially flat.

8. The apparatus of claim 1, wherein the side wall of the shell being substantially cylindrical, and the distal wall of the shell being substantially hemispherical.

9. The apparatus of claim 1, wherein the distal tip of the thermocouple is potted in an electrically insulating and thermally conductive material.

10. The apparatus of claim 9, further comprising a thin walled tube surrounding at least a distal portion of the thermocouple.

11. The apparatus of claim 10, wherein the electrically insulating and thermally conductive material is an epoxy and the tube is a thin-walled Polyimide tube.

12. The apparatus of claim 1, wherein a temperature sensing element is positioned in the range of about 0.1 mm to about 4 mm from a distal tip or distal end of the thermocouple.

13. The apparatus of claim 1, where in a temperature sensing element is positioned at about 0.7 mm from a distal tip or distal end of the thermocouple.

14. A medical instrument, comprising:
a steerable irrigated ablation catheter having a proximal end and a distal ablation tip;
a fluid reservoir located in a distal portion of the steerable irrigated ablation catheter;
and
a thermocouple positioned within the steerable irrigated ablation catheter and disposed from the proximal end to the distal portion of the irrigated ablation catheter, wherein a distal tip of the thermocouple being positioned proximal of the fluid reservoir, the thermocouple being electrically isolated from the ablation tip.

15. The instrument of claim 14, further comprising:
a steerable sheath having a working lumen, wherein at least a portion of the steerable irrigated ablation catheter being slideably disposed within the working lumen of the steerable sheath.

16. The instrument of claim 15, further comprising a wire extending from the proximal end to the distal portion of the steerable irrigated ablation catheter, wherein a distal end of the wire being connected to the distal ablation tip.

17. The instrument of claim 14, wherein the distal ablation tip has a substantially cylindrical side surface and a substantially flat distal surface.

18. The instrument of claim 14, wherein the distal ablation tip has a substantially cylindrical side surface and a substantially hemispherical distal surface.

19. The apparatus of claim 14, wherein the distal tip of the thermocouple is potted in an electrically insulating and thermally conductive material.

20. The apparatus of claim 19, wherein the electrically insulating and thermally conductive material is an epoxy and the tube is a thin-walled Polyimide tube.
